(19) United States
(12) Patent Application Publication
(10) Pub. No.: US 2008/0319445 A9
(43) Pub. Date: Dec. 25, 2008

(54) Apparatus and methods for delivering compounds into vertebrae for vertebroplasty

(75) Inventors: Scott McGill, San Ramon, CA (US); Harold F. Carrison, Pleasanton, CA (US); Mukund R. Patel, San Jose, CA (US); Albert Arcadio Delacruz, Colma, CA (US)

Correspondence Address: VISTA IP LAW GROUP LLP, 12930 Saratoga Avenue, Suite D-2, Saratoga, CA, 95070, US

(73) Assignee: Scimed Life Systems, Inc., Maple Grove, MN (US)

(21) Appl. No.: 10/956,249

(22) Filed: Sep. 30, 2004

Related U.S. Application Data
(63) Continuation-in-part of application No. 10/920,581, filed on Aug. 17, 2004.

(51) Int. Cl. A61F 2/34

(52) U.S. Cl. 606/92

(57) ABSTRACT

An apparatus for delivering bone cement into a vertebra, includes a cannula, a delivery device in communication with the cannula and a pressure delivery device in communication with the delivery device. The pressure delivery device provides an actuating force that acts either directly or through a medium to cause a flowable compound to be delivered from the delivery device to the cannula and into the vertebra. The pressure delivery device causes a pressurized compound to be delivered, the pressurized compound may be liquid or gaseous CO₂ or other mediums.

### BACKGROUND OF THE INVENTION

1. Field of the Invention

The invention relates generally to apparatus and methods for delivering compounds into a body, and more particularly to apparatus and methods for delivering bone cement, biomaterials, and/or other flowable compounds into vertebrae, e.g., during a vertebroplasty procedure.

2. Background of the Invention

Vertebroplasty is a procedure during which bone cement, biomaterials, and/or other flowable compounds are delivered into a vertebra. A delivery syringe or other delivery device is generally provided within which the bone cement to be delivered is stored shortly before the bone cement is to be delivered. For example, the delivery device may include a barrel or housing including an open inlet end and an exit end with a narrow outlet. A plunger or threaded driver may be advanced into the inlet end to force bone cement within the barrel out the outlet in the exit end.

A cannula may be inserted percutaneously through the cutaneous layers of tissue above a hard tissue structure being treated and into the hard tissue structure. For example, the hard tissue structure may be a vertebra, and the cannula may include a sharpened tip to penetrate through cortical bone and into the cancellous bone within the vertebra. Alternatively, the hard tissue structure may be exposed using conventional surgical procedures before inserting the cannula and/or the cannula may be inserted over a needle previously placed or simultaneously advanced into the vertebra.

A semi-rigid or flexible tube, e.g., twenty to fifty centimeters long, may be connected between the proximal end of the cannula and the outlet of the delivery device to deliver bone cement via the tube into the hard tissue structure, e.g., to keep the user's hands and/or the delivery device out of the field of an imaging device, such as a fluoroscope, that may be used to monitor the procedure. The tube may be bent slightly during the procedure to lessen the stress that on the cannula and to aid in ensuring the user's hands and/or the delivery device is kept out of the field of an imaging device that may be used during the procedure.

Alternatively, the delivery syringe may be connected directly to the proximal end of the cannula. Such a rigid connection, however, requires a user to support the delivery syringe/cannula combination, which may expose the user to x-ray radiation, e.g., from a fluoroscope used to monitor the injection of the material as it is being injected, requiring the user to wear appropriate additional x-ray protection, which may be cumbersome, inconvenient, and ineffective.

In addition, because of the high viscosity of bone cement, high pressures are generally required to inject bone cement from the delivery device, through the tube and cannula, and into the hard tissue structure. For example, pressures of up to one to three thousand pounds per square inch (1,000-3,000 psi) may be required to inject bone cement from the delivery device. This requires the user to apply substantial force, while simultaneously supporting the weight of the delivery device and its contents. This may cause fatigue of the user and/or undesired movement of the cannula delivery device during the procedure

A variety of apparatus and methods for delivering bone cement have been disclosed. Such devices are disclosed in U.S. patent application Ser. Nos. 10/463,757 filed on Jun. 17, 2003, and Ser. No. 10/920,581 filed on Aug. 17, 2004, which are hereby incorporated by reference in their entirety for all purposes as if fully set forth herein.

Accordingly, additional apparatus and methods for delivering bone cement or other compounds into vertebrae would be useful.

### SUMMARY OF THE INVENTION

The invention is directed to apparatus and methods for delivering compounds into a body, and more particularly to apparatus and methods for delivering bone cement, biomaterials, and/or other flowable compounds into vertebrae, e.g., during a vertebroplasty procedure.

In one embodiment, the apparatus includes a cannula sized and shaped for insertion into a vertebra. The cannula has a proximal end and a distal end, both the proximal end and the distal end are open and a lumen extends therethrough. The apparatus also includes a delivery device with a barrel defining a cavity for receiving a flowable compound, and a distal end having an outlet in fluid communication with the cavity. The outlet, is pivotally connected to the proximal end of the cannula so that the outlet communicates with the lumen of the cannula. The apparatus further includes a pressure delivery device in communication with the delivery device. The pressure delivery device provides an actuating force that acts upon the flowable compound. The actuating force may act directly upon the flowable compound.

In another embodiment, the actuating force may act indirectly on the flowable compound. For example, the actuating force may act upon a piston disposed between the pressure delivery device and the flowable compound.

In another embodiment, the actuating force acts upon a piston configured to translate the actuating force through a medium to the flowable compound. The intermediary medium may be saline.

In another embodiment, the actuating force acts upon a first piston configured to translate the actuating force through a medium to the flowable compound.

The apparatus may further include a trigger. The trigger may be connected to the pressure delivery device, where the trigger is configured to control the actuating force.

In another embodiment, the apparatus has a value that controls the actuating force in addition to a trigger. The position of the trigger determines an associated position of the valve. For example, a first position of the trigger may open the valve, and a second position of the trigger may the close the valve. Alternatively, a first position of the trigger may open a valve and direct the actuating force in a first direction, and a second position of the trigger may open the valve and direct the actuating force in a second direction.

In another embodiment, a valve connected to the pressure delivery device controls the direction of the actuating force.

The actuating force may be CO₂ or liquid CO₂.

In another embodiment, the apparatus includes a cannula sized and shaped for insertion into a vertebra. The cannula has a proximal end and a distal end, both the proximal end and the distal end are open and a lumen extends therethrough. The apparatus also includes a delivery device with a barrel defining a cavity for receiving a flowable compound, and a distal end having an outlet in fluid communication with the cavity. The outlet, is pivotally connected to the proximal end of the cannula so that the outlet communicates with the lumen of the cannula. The apparatus further includes a pressure delivery device in communication with the delivery device. The pressure delivery device provides a gaseous actuating force that acts upon the flowable compound. A trigger is connected to the pressure delivery device, to control the gaseous actuating force.

In another embodiment, the gaseous actuating force may be CO₂. Alternatively, the gaseous actuating force may be liquid CO₂.

In another embodiment, the apparatus is a pressure delivery system. The pressure delivery system includes a canister configured to hold a pressurized compound, a valve connected to the canister and configured to control the pressurized compound and a trigger integral to the valve. The trigger directs the pressurized compound. As with other embodiments, the pressurized compound may be CO₂ or liquid CO₂.

The invention also includes a method for delivering bone cement into a vertebra. The method includes inserting a cannula into a vertebra; providing an actuating force from a pressure delivery device to a flowable compound, and delivering the flowable compound to the cannula and into the vertebra. The actuating force controls the movement of the flowable compound

Other objects and features of the invention will become apparent from consideration of the following descriptions taken in conjunction with the accompanying drawings.

### BRIEF DESCRIPTION OF THE DRAWINGS

The drawings illustrate the design and utility of embodiments of the invention, in which similar elements are referred to by common reference numerals and in which:

FIG. 1 is a partial cross-sectional side view of an embodiment of an apparatus for delivering bone cement into a vertebra, in accordance with the invention.

FIG. 2 is a partial cross-sectional side view of another embodiment of an apparatus for delivering bone cement into a vertebra, in accordance with the invention.

FIG. 3 is a partial cross-sectional side view of yet another embodiment of an apparatus for delivering bone cement into a vertebra, in accordance with the invention.

FIG. 4 is a partial cross-sectional side view of still another embodiment of an apparatus for delivering bone cement into a vertebra in accordance with the invention.

### DETAILED DESCRIPTION OF THE ILLUSTRATED EMBODIMENTS

Various embodiments of the invention are described hereinafter with reference to the figures. It should be noted that the figures are not drawn to scale and elements of similar structures or functions are represented by like reference numerals throughout the figures. It should also be noted that the figures are only intended to facilitate the description of specific embodiments of the invention. They are not intended as an exhaustive description of the invention or as a limitation on the scope of the invention. In addition, an aspect described in conjunction with a particular embodiment of the invention is not necessarily limited to that embodiment and can be practiced in any other embodiments of the invention.

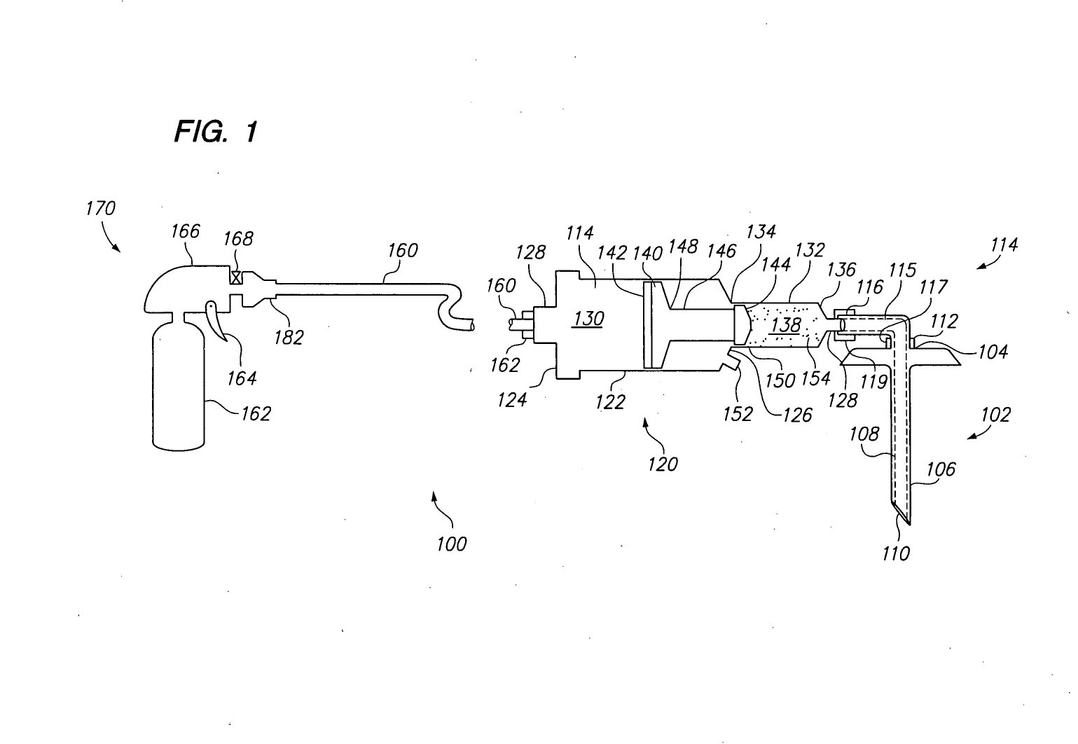

Turning to the drawings, FIG. 1 shows an embodiment of an apparatus 100 for delivering bone cement, biomaterial, and/or other compounds into a vertebra or other hard tissue structure (not shown). Generally, the apparatus 100 includes a cannula 102, a delivery syringe or other delivery device 120, a pivot fitting 114 for pivotally connecting the cannula 102 to the delivery syringe 120, a pressure delivery device 170, and a tubing 160 for connecting the pressure delivery device 170 to the delivery syringe 120.

Generally, the cannula 102 is a substantially rigid elongate tubular member including a proximal end 104, a distal end 106, and a lumen 108 extending therethrough. The cannula 102 may be a needle, i.e., including a beveled or otherwise sharpened distal tip 110 such that the distal end 106 may penetrate into hard tissue, such as bone, although alternatively the cannula 102 may have a substantially blunt distal tip (not shown) and initial access into the hard tissue may be made through other means with the cannula 102 being inserted thereafter. A cannula connector 112 such as a luer fitting may be provided at the proximal end 104 for attaching the cannula 102 to a pivot fitting 140, as described further below.

The cannula 102 may have a substantially uniform diameter or cross-section, similar to known needles for accessing a vertebra, e.g., between about eleven and thirteen gauge (11-13 GA). Alternatively, the cannula 102 may taper from the proximal end 104 at least partially towards the distal end 106, e.g., such that the distal end 106 corresponds to a conventional needle diameter. The cannula 102 may be formed from conventional materials, e.g., stainless steel, metals, plastics, and laminated tubes.

The pivot fitting 114 pivotally connects the cannula 102 to the delivery syringe 120. The pivot fitting 114 allows the delivery syringe 120 to rotate inline with the central axis of the cannula 102 to assist in the placement of the delivery syringe 120 in a location relative to a treatment site that is best suited to minimize interference with the procedure. The pivoting fitting 114 may also allow the delivery syringe 120 to rotate transverse to a central axis of the pivot fitting 114 to provide for ease of connection of the delivery syringe 120 to the pivot fitting 114 and to assist in the placement of the delivery syringe 120 at a suitable angle relative to a body surface thereby minimizing the stress place on the cannula 102 as a result of the weight of the delivery syringe 120. The pivot fitting 114 may be comprised of multiple components that are assembled; alternatively the pivot fitting 114 may be constructed as a single component.

The pivot fitting 114 has a lumen 115 that extends from a proximal end 116 to a distal end 117 of the pivot fitting 114. The lumen 115 provides a substantially fluid-tight passage that extends from the proximal end 116 to the distal end 117 of the pivot fitting 114, allowing for an unobstructed connection between the delivery syringe 120 and the cannula 102. The pivot fitting 114 may be formed from any variety of materials, known to those of skill in the art, capable of handling the internal pressures experienced when bone cement is delivered, e.g., between about one and three thousand pounds per square inch (1,000-3,000 psi). In addition, the pivot fitting 114 should be sufficiently strong to support any bending or other forces experienced when the pivot fitting 114 is used to connect a cannula 102 to a delivery syringe 120 during a vertebroplasty procedure.

In alternative embodiments, the pivot fitting 114 may be substantially permanently attached to at least one of the cannula 102 or the delivery syringe 120. For example, the pivot fitting 114 may be provided as part of the delivery syringe 120, i.e., extending from a distal end 136 of the delivery syringe 120, thereby eliminating connector 119 between the pivot fitting 114 and the delivery syringe 120. In this instance, therefore, the distal end 117 of the pivot fitting 114 may have a connector 112, for connection to the cannula 102. Alternatively, the pivot fitting 114 may be substantially permanently attached to the proximal end 104 the cannula 102 and a connector implemented for coupling the delivery syringe 120 to the pivot fitting 114. Thus, one or both ends of the pivot fitting 120 may be detachable from and/or substantially permanently attached to the cannula 102 and/or delivery syringe 120.

A variety of such pivot fittings are known, for example U.S. patent application Ser. No. 10/716,641 describes a pivot fitting for coupling a delivery syringe to a cannula. U.S. patent application Ser. No. 10/920,581 describes a pivot fitting for coupling a delivery syringe to a cannula where the pivot fitting may rotate about two different axis.

With continued reference to FIG. 1, the delivery syringe 120 generally includes a first barrel 122 including a proximal end 124, a distal end 126, and fluid communication port 128, thereby defining a first interior space or cavity 130 and a second barrel 132 including a proximal end 134 and a distal end 136 thereby defining a second interior space or cavity 138.

A first piston 140 may be slidably disposed within the first cavity 130 of the first barrel 122. Preferably the proximal end 124 of the first barrel 122 is constructed so as to substantially seal the first barrel 122 leaving only the fluid communication port 128 open. The first piston 140 may be advanced distally, toward the distal end 126 of the first barrel 122 by applying a pressure to a proximal end 142 of the first piston 140. A second piston 144 may be slidably disposed within the second cavity 138 of the second barrel 132. Preferably a piston rod 146 is connected to a distal end 148 of the first piston 140. The piston rod 146 extends from the distal end 148 of the first piston 140 and is connected to a proximal end 150 of the second piston 144. When the first piston 140 advances, the piston rod 146 exerts a force on the second piston 144, causing the second piston 144 to also advance.

The first barrel 122 may be constructed to include a vent 152 toward the distal end 126 of the first barrel 122. The vent 152 allows excess pressure that builds up in the first cavity 130 to be released as the first piston 140 slides toward the distal end 126 of the first barrel 122. This release of pressure facilitates the movement of the first piston 140.

The second piston 144 may be used to exert pressure on bone cement or other flowable materials contained within the second cavity 138 of the delivery syringe 120 so that the bone cement may be delivered into a vertebra or other bone structure. The pressure may be created by delivering a pressurized compound, for example CO₂ gas or liquid CO₂ through the fluid communication port 128 (as discussed below) into a proximal section of the first cavity 130. As a result of the pressure, the first piston 140 may be advanced distally to cause the piston rod 146 and the second piston 144 to similarly advance distally. Generally, the cross section of the first piston 140 must be greater than the cross section of the second piston 144 so that the pressure will increase as the first piston 140 and the second piston 144 move distally. In one embodiment, the cross section of the first piston 140 is at least 1.05 times larger than the cross section of the second piston 144 and the cross section of the first piston 140 is not more than 10.05 times larger than the cross section of the second piston 144. In another embodiment, the cross section of the first piston 140 is up to 100 times larger than the cross section of the second piston 144.

In this embodiment, the delivery syringe cross-section, and the piston cross-section decrease between the first and the second barrel. As a result of this geometry, the pressure is multiplied, thereby allowing a lower pressure to be exerted at the proximal end 142 of the first piston 140 while still providing adequate pressure at the distal end 136 of the second barrel 132 to force the bone cement through the delivery syringe 120.

The delivery syringe 120 may be constructed from any materials known to those of skill in the art, for example, the delivery syringe 120 may be constructed from Cyclic Olefin Copolymers (COC), Polycarbonate, Polystyrene, plastics, metals, or any variety of surgical metals.

Continuing with FIG. 1, pressure is delivered to the delivery syringe 120, by means of a pressure deliver system 170. The pressure delivery system generally comprises a canister configured to hold a pressurized compound 162, a trigger 164, a pressure valve 166, and a blow off valve 168.

The canister 162 is attached to the pressure valve 166 that controls the release of the pressurized compound, such as liquid CO₂, into the delivery system. The connection between the canister 162 and the pressure valve 166 must create an airtight seal, for example, a threaded connection may be used. When the trigger 164 is depressed, the pressure valve 166 is opened and the pressurized compound flows through the pressure valve 166, is pressurized, and released into the system. When the trigger 164 is released, the pressurized compound is allowed to escape through the blow-off valve 168, and is no longer delivered to the system, thereby not further pressurizing the system. When the pressure in the system is released, the flowable compound ceases flowing from the outlet port 128 in the distal end of the second barrel 132 and delivery of the flowable compound to the cannula 102 and into the vertebra is stopped.

Alternatively, the pressure valve may be configured with a manually controlled blow-off valve (not shown). If configured in this manner, the pressure in the system is not released when the trigger 164 is released, but instead, the system remains pressurized. The pressure slowly diminishes as the flowable compound is dispensed. If operated in this manner, the system will continue to deliver the flowable compound through the cannula 102 and into the vertebra until the pressure in the system is dispersed.

Connection of the pressure delivery system 170 to the delivery syringe 120 may be made through a connector 162 attached to the opening 128 on the delivery syringe 120 that mates with the connector (not shown) on a tubing 160. The tubing 160 is then connected to the pressure delivery system 170 through a connector 182. The pressure delivery system 170 delivers the pressurized compound through the tubing 160 into the first cavity 130 of the first barrel 122 to cause the first piston 140 to slide distally within the first cavity 130. The tubing 160, and opening 128 may include integral connectors as opposed to connectors as described above. Alternatively, the tubing 160 may be substantially permanently attached to the delivery syringe 120.

The tubing 160 may vary from being a semi-rigid elongated member to being a relatively compliant flexible tube. For example the tubing may be polyurethane, or braid or coil reinforced catheter materials, PEEK or polyamide or metal. The tubing 160 preferably has sufficient length such that a proximal end 164 of the tubing 160 may be disposed away from a patient, and preferably away from a field of an imaging device, e.g., fluoroscope. For example, the tubing 160 may have a length between about ten and seventy centimeters (10-70 cm). Furthermore, the tubing 160 must have sufficient cross-sectional strength to withstand the delivery pressures as described above.

In order to deliver bone cement or other biomaterials, the cannula 102 must be inserted into the vertebra (not shown). If the distal end 106 of the cannula 102 includes a sharpened distal tip 110, the distal tip 110 may be inserted directly into a vertebra, e.g., until the distal end 106 penetrates the cortical bone and enters the cancellous bone region therein. The cannula 102 may be inserted percutaneously, e.g., through cutaneous fat, muscle, and/or other tissue overlying the vertebra. Alternatively, the vertebra may be at least partially exposed before inserting the cannula 102, e.g., using an open surgical procedure. For example, the tissue overlying the vertebra may be surgically dissected and/or retracted to expose the vertebra, and the distal end 106 of the cannula 102 may be inserted into the exposed vertebra.

In one embodiment (if the cannula 102 is initially separate from the pivot fitting 114 and/or the delivery syringe 120), a stylet, an obturator or other device (not shown) may be inserted into the lumen 108 of the cannula 102 to prevent tissue and/or fluid, such as blood, from entering the lumen 108 while the cannula 102 is advanced through tissue. In a further alternative, a stylet and sheath (also not shown) may be percutaneously inserted through overlying tissue to access the vertebra. The stylet may be removed from within the sheath, and the cannula 102 may be advanced through the sheath and then inserted into the vertebra.

It will be appreciated that any known open or minimally invasive procedure may be used to place the cannula 102 into the vertebra. In addition, it will be appreciated that the insertion of the cannula 102 may be monitored using external imaging, such as fluoroscopy, ultrasound imaging, magnetic resonance imaging (“MRI”), and the like (not shown). For example, the cannula 102 may be formed from radiopaque material and/or may include one or more radiopaque markers to facilitate monitoring the position of the cannula 102 as it is advanced into the vertebra using a fluoroscope, as is known in the art.

Once the distal end 106 of the cannula 102 is inserted into the vertebra the delivery syringe 120 (with bone cement or other compound provided therein using conventional methods) may be connected to the proximal end 104 of the cannula 102. For example, the pivot fitting 114 may be connected first (or, alternatively, may be substantially permanently attached) to the distal end 136 of the delivery syringe 120, for example, the outlet port 128. The loose end of the pivot fitting 114 may be connected to the proximal end 104 of the cannula 102, e.g., by connecting mating luer lock connectors (only 112 shown).

Alternatively, the pivot fitting 114 may be substantially permanently attached to the proximal end 104 of the cannula 102, and then may be attached to the distal end 136 of the delivery syringe 120, e.g., using mating luer lock connectors (only 119 shown). In a further alternative, the pivot fitting 114 may be substantially permanently attached to both the cannula 102 and the delivery syringe 120 (not shown), such that the delivery syringe 120 is attached to the cannula 102 when the cannula 102 is inserted into the vertebra.

Once the apparatus 100 is assembled, the delivery syringe 120 may be disposed at a desired angle relative to the cannula 102. For example, it may be desirable to lay the delivery syringe 120 directly on the patient's skin (e.g., on the patient's back) overlying the vertebra or alternatively to support the delivery syringe 120 by a stand so that an optimal angle, relative to the patients skin is obtained.

Because the delivery syringe 120 may be located within the field of an imaging system, e.g., a fluoroscope (not shown), it may be desirable to extend the tubing 160 away from the patient's body, until the pressure delivery system 170 is located outside the field of the imaging system. This will remove the operator away from the field, thereby substantially reducing his exposure to radiation and the like.

Once the delivery syringe 120 is disposed at a desired location, the pressure delivery system 170 may be engaged to deliver the bone cement or other compound from the delivery syringe 120 through the pivot fitting 114 and the cannula 102 into the cancellous bone region of the vertebra (as described previously). Because the path through which the bone cement passes is substantially shorter than the path when conventional tubing is used to connect a delivery syringe to a cannula (not shown), less pressure may be required to deliver the bone cement than using such tubing systems. In addition, less bone cement may be wasted, because the flow path may have less volume that must be filled with bone cement before the bone cement exits the cannula 102 and enter the vertebra.

Once sufficient bone cement is delivered into the vertebra, the cannula 102 may be removed and the puncture or other access opening may be closed using conventional procedures.

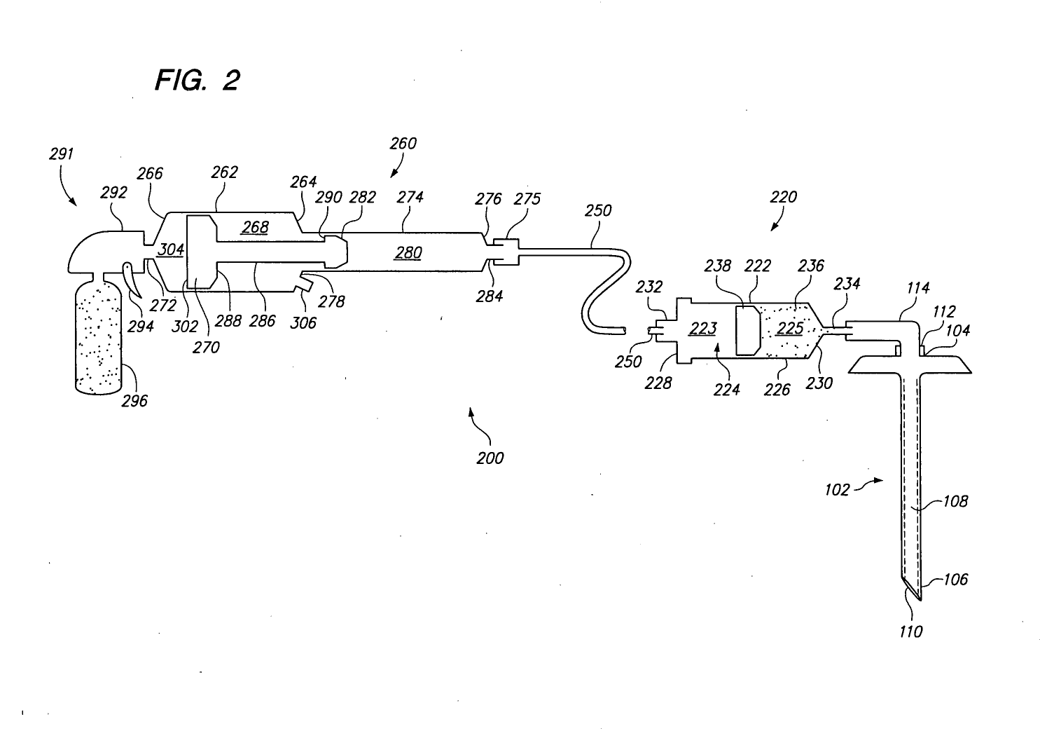

FIG. 2 shows an embodiment of an apparatus 200 for delivering bone cement, biomaterial, and/or other compounds into a vertebra or other hard tissue structure (not shown). Generally, the apparatus 200 includes a cannula 102, a delivery syringe or other delivery device 220, a pivot fitting 114 for pivotally connecting the cannula 102 to the delivery syringe 220, an actuator 260 connected to the delivery syringe 220 through a tubing 250 for delivering pressure to the delivery syringe 220, and a pressure delivery device 291, that provides pressure to activate the actuator 260.

A pivot fitting 114 (such as the pivot fitting described in FIG. 1) pivotally connects a cannula 102 (such as the cannula described in relation to FIG. 1) to the delivery syringe 220. The delivery syringe 220 generally includes a barrel 222 including a proximal end 228, and a distal end 230, thereby defining an interior space or cavity 223. The interior cavity may generally be described as having two sections, a proximal end cavity 224 and a distal end cavity 225. Within the distal end cavity a flowable compound 236, such as bone cement and/or biomaterials may be contained. A slidable piston 238 may initially be contained within the proximal end cavity 224. The distal end 230 of the delivery syringe 220 may include an outlet port 234 that is in fluid communication with the cavity 224, and more specifically with the distal end cavity 225. A luer lock or other connector (not shown) may be provided on the outlet port 234 for cooperating with a complementary connector (not shown) on the pivot fitting 114.

The piston 238 slidably disposed initially in the proximal end cavity 224 of the barrel 222 is designed to force a flowable compound 236 within the distal end cavity 225 out through the outlet port 234. The piston 238 may be advanced distally, as described below, thereby applying a force creating sufficient pressure to push the flowable compound 236 within the distal end cavity 225 out the outlet port 234. Optionally, the piston 238 may include a nipple (not shown) extending into the distal end cavity 225. The nipple may have a size corresponding to the outlet port 234 of the delivery syringe 220, e.g., such that the nipple may be slidably received in the outlet port 234 as the piston 238 is slidably forced toward the distal end 230. The nipple may minimize the amount of bone cement remaining within the delivery syringe 220 when the piston 238 has reached the distal end 230 of the barrel 222. Furthermore, the piston 238 may include gaskets (not shown) such as o-rings designed to ensure a tight seal between the piston 238 and the barrel 222 while also preventing any contamination of the flowable compound 236 that is located in the distal end cavity 225, with a fluid or gas that may be located on the input pressure or hydraulic side near the proximal end cavity end 223.

Preferably, the proximal end 228 of the barrel 222 is substantially closed but includes an opening 232 through which an actuating device 260, may be connected to the barrel 222, for delivering a fluid, or gas into the proximal end cavity 224. Connection of the actuating device 260 to the delivery syringe 220 may be made through a connector (not shown) attached to the opening 232 on the delivery syringe 220 that mates with the connector also not shown) on a tubing 250. The tubing 250 is then connected to the actuating device through a connector 275.

The actuating device 260 delivers a pressurized noncompressible liquid such as saline through the tubing 250 into the proximal end cavity 223 to cause the piston 238 to slide distally within the cavity 224 and towards the distal end cavity 225. The saline is initially contained within a second cavity 280 (described below) of the actuating device 260. The tubing 250, and opening 232 may include integral connectors as opposed to connectors as described above. Alternatively, the tubing 250 may be substantially permanently attached to either or both the delivery syringe 220 or the actuator 260.

The actuating device 260 is similar to the delivery syringe 120 described in conjunction with FIG. 1. However, unlike the delivery syringe 120 of FIG. 1, the actuating device does not contain the flowable compound in a second barrel, but instead contains saline or another non-compressible fluid. The actuating device 260 generally includes a first barrel 262 including a proximal end 266, a distal end 264, and fluid communication port 272, thereby defining a first proximal cavity 304 and a first distal cavity 268, and a second barrel 274 including a proximal end 278, a distal end 276 and an outlet port 284 thereby defining a second cavity 280. A first piston 270 may be slidably disposed within the first proximal cavity 304 of the first barrel 262. Preferably the proximal end 266 of the first barrel 262 is constructed so as to substantially seal the first barrel 262 leaving only the fluid communication port 272 open. The first piston 270 may be advanced distally, toward the distal end 264 of the first barrel 262 by applying a pressure to a proximal end 302 of the first piston 270. A second piston 282 may be slidably disposed within the second cavity 280 of the second barrel 274. Preferably a piston rod 286 is connected to a distal end 288 of the first piston 270. The piston rod 286 extends from the distal end 288 of the first piston 270 and is connected to a proximal end 290 of the second piston 282. When the first piston 270 advances, the piston rod 286 exerts a force on the second piston 282, causing the second piston 282 to also advance. As the second piston advances, the saline or other noncompressible fluid is forced out the outlet port 284, through the tubing 250 and into the proximal end cavity 223 of the delivery syringe 220.

The first barrel 262 may be constructed to include a vent 306 toward the distal end 264 of the first barrel 262. The vent 306 allows excess pressure that builds up in the first distal cavity 268 to be released as the first piston 270 slides toward the distal end 264 of the first barrel 262. This release of pressure facilitates the movement of the first piston 270.

As a result of the flow of the saline from the actuator 260, through the tubing 250 and into the delivery syringe 220, pressure is exerted on the piston 238 in the delivery syringe 220. This pressure causes the piston 230 to move distally forcing the flowable compound 236 through the outlet port 234, through the pivot fitting 114 and cannula 102 and into the vertebra or other bone structure. The pressure may be created by delivering a pressurized compound through the fluid communication port 272 on the actuating device 260 (as discussed below) into the first proximal cavity 304 of the first barrel 262 of the actuating device 260. As a result of the pressure, the first piston 270 may be advanced distally to cause the piston rod 286 and the second piston 282 to similarly advance distally. Since the cross section of the second piston 282 is smaller than the cross section of the first piston 270, the pressure exerted by the second piston 282 will be greater that the pressure exerted by the first piston 282 (as discussed previously in relation to FIG. 1)

Continuing with FIG. 2, pressure is delivered to the actuator 260, by means of a pressure deliver system 291. The pressure delivery system 291 generally comprises a canister configured to hold a pressurized compound 296, a trigger 294, a pressure valve 292, and optionally a blow off valve (not shown).

The canister 296 is attached to the pressure valve 292 that controls the release of the pressurized compound into the actuator 260 as discussed previously. When the trigger 294 is depressed, the pressure valve 292 is opened, the pressurized compound is allowed to flow through the pressure valve 292, is pressurized, and released into inlet port 272 at the proximal end 266 of the first barrel 262 of the actuator 260. When the trigger 294 is released, the pressurized compound is allowed to escape through a blow-off valve and is no longer delivered to the system, thereby releasing the pressure in the system. When the pressure is released, the actuator 260 ceases to force the saline into the opening 232 at the proximal end 238 of the delivery syringe 220 and therefore, the flowable compound ceases flowing from the outlet port 234 in the distal end 230 of the delivery syringe 220 through the pivot fitting 140, though the cannula 102 and into the vertebrae.

Alternatively, the pressure valve could be configured with a manually controlled blow-off valve (not shown). If configured in this manner, the pressure in the system is not released when the trigger 294 is released, but instead, the system remains pressurized. The pressure slowly diminishes as the flowable compound is dispensed. If operated in this manner, the system will continue to deliver the flowable compound through the cannula 102 and into the vertebrae until the pressure in the system is reduced.

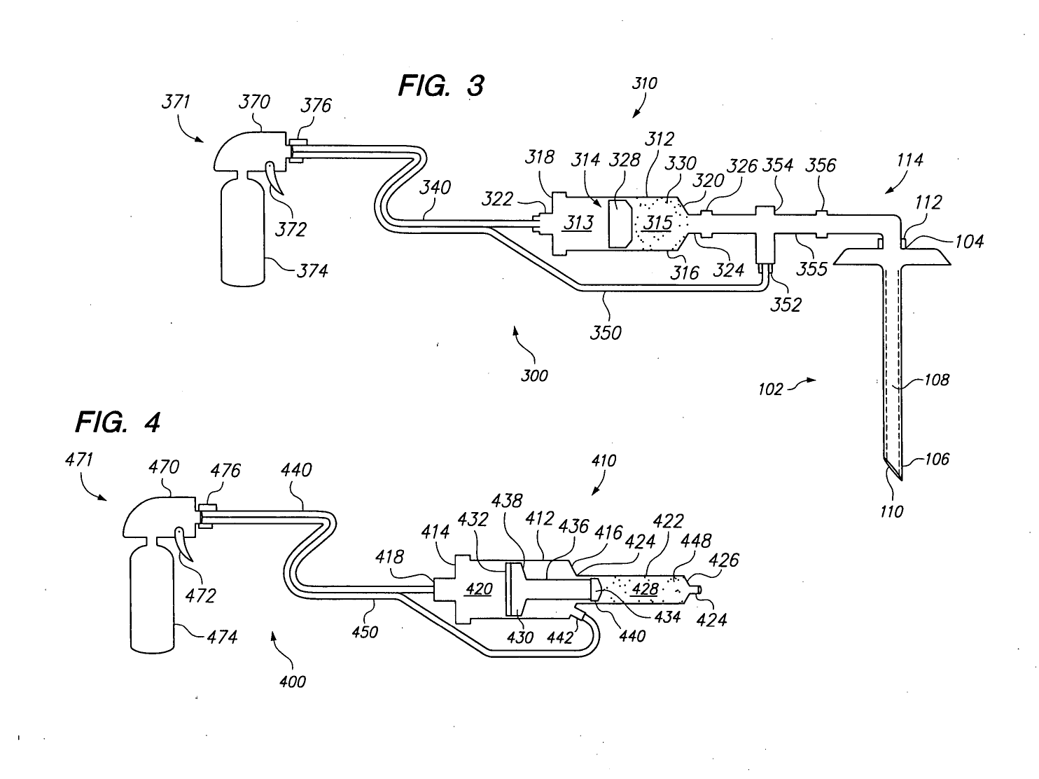

With reference now to FIG. 3, FIG. 3 shows another embodiment of an apparatus 300 for delivering bone cement, biomaterial, and/or other compounds into a vertebra or other hard tissue structure (not shown). Generally, the apparatus 300 includes a cannula 102, a delivery syringe or other delivery device 310, a pivot fitting 114 for pivotally connecting the cannula 102 to the delivery syringe 310, a pressure delivery device 371, a first tubing 340 for connecting the pressure delivery device 371 to an opening 322, and a second tubing 350 for connecting the pressure delivery device 371 to valve 354 located between the delivery syringe 310 and the pivot fitting 114.

A pivot fitting 114 (such as the pivot fitting described in FIG. 1) pivotally connects a cannula 102 (such as the cannula described in relation to FIG. 1) to the delivery syringe 310. The delivery syringe 310 generally includes a barrel 312 including a proximal end 318, and a distal end 320 thereby defining an interior space or cavity 314. The interior cavity 314 may generally be described as having two sections, a proximal end cavity 313 and a distal end cavity 315. Within the distal end cavity a flowable compound, such as bone cement and/or biomaterials (not shown), may be contained. The distal end 320 of the delivery syringe 310 may include an outlet port 324 that is in fluid communication with the cavity 314 and specifically with the distal end cavity 315. A luer lock or other connector (not shown) may be provided on the outlet port 324 for cooperating with a complementary connector (also not shown) on a rigid tubing 355. The rigid tubing 355 connects the valve 354 to the delivery syringe 310 and the pivot fitting 114.

A piston 328 may be slidably disposed initially in the proximal end cavity 313 of the barrel 312 for forcing a flowable compound 330 within the barrel 222 out through the outlet port 326. The piston 328 may be advanced distally, as described below, thereby applying a force creating sufficient pressure to push the flowable compound 330 in the distal end cavity 315 out the outlet port 324.

Preferably, the proximal end 318 of the barrel 312 is substantially closed but includes an opening 322 through which a pressure delivery device 371 may be connected to the delivery syringe 310, for delivering a pressurized compound into the proximal end cavity 313 of the barrel cavity 314.

Pressure is delivered to the delivery syringe 310, by means of a pressure deliver system 371. The pressure delivery system generally comprises a canister configured to hold a pressurized compound 374, a trigger 372, and a pressure valve 370.

The canister 374 is attached to the pressure valve 370 that controls the release of the pressurized compound into the delivery system as described previously. When the trigger 372 is depressed, the pressure valve 370 is opened, the pressurized compound is allowed to flow through the pressure valve 370, is pressurized, and released into the first tubing 340 and the second tubing 350. The first tubing 340 connects the pressure delivery system 371 to the delivery syringe 310. The connection may be made through a connector (not shown) attached to the opening 322 on the delivery syringe 310 that mates with a connector (also not shown) on the first tubing 340. Alternatively the first tubing 340 may be permanently affixed to either or both the pressure delivery system 371 and the delivery syringe 310. The second tubing 350 is connected to the valve 354. The valve 354 may be a pneumatic valve that is spring loaded. When the pressurized compound is released into the second tubing 350 the pressure applied opens the valve 354. Therefore the flowable compound is allowed to flow through the pivot fitting 114, through the cannula 102 and into the vertebra.

When the trigger 372 is released, the pressurized compound is no longer delivered to either the first or the second tubing 340, 350. The first tubing 340 therefore no longer provides pressurization to the delivery syringe 310 and as the pressure in the system is released, the flowable compound ceases flowing from the outlet port 324 in the distal end 320 of the delivery syringe 310. Furthermore, since there is similarly no pressure in the second tubing 350, the valve 354 is closed and delivery through the cannula 102 and into the vertebrae is stopped.

In an alternative embodiment of the apparatus 300 of FIG. 3, a third tubing is provided. The third tubing is connected to the valve 354 opposite the second tubing 350. When configured in this manner, the second tubing 350 and the third tubing control the valve 354. The second tubing 350 when pressurized opens the valve 354. The third tubing when pressurized closes the valve 354. In this example, the valve need not be spring-loaded. The flow of the pressurized compound into the second and third tubings may be controlled by a switch on the pressure valve 370, or by an additional valve. The first tubing 340 remains configured as described previously.

Turning now to FIG. 4, FIG. 4 shows still another embodiment of an apparatus 400 for delivering bone cement, biomaterial, and/or other compounds into a vertebra or other hard tissue structure (not shown). Generally, the apparatus 400 includes a cannula (not shown), a delivery syringe or other delivery device 410, a pivot fitting (not shown) for pivotally connecting the cannula to the delivery syringe 410, a pressure delivery device 471, a first tubing 440 for connecting the pressure delivery device 471 to an opening 422 in the delivery syringe 410, and a second tubing 450 for connecting the pressure delivery device 471 to a port 426 located on the distal end 420 of the delivery syringe 410.

In operation, a pivot fitting (such as the pivot fitting described in FIG. 1) pivotally connects a cannula (such as the cannula described in relation to FIG. 1) to the delivery syringe 410. The delivery syringe 410 generally includes a first barrel 412 including a proximal end 414, a distal end 416, and fluid communication port 418, thereby defining a first interior space or cavity 420 and a second barrel 422 including a proximal end 424 and a distal end 426 thereby defining a second interior space or cavity 428.

A first piston 430 may be slidably disposed within the first cavity 420 of the first barrel 412. Preferably the proximal end 414 of the first barrel 412 is constructed so as to substantially seal the first barrel 412 leaving only the fluid communication port 418 open. A pressure delivery device 471 may be connected to the first barrel 412, by means of a first tubing 440, connected to the fluid communication port 418. The first piston 430 may be advanced distally, toward the distal end 416 of the first barrel 412 by applying a pressure to a proximal end 432 of the first piston 430. A second piston 434 may be slidably disposed within the second cavity 428 of the second barrel 422. Preferably a piston rod 436 is connected to a distal end 438 of the first piston 430. The piston rod 436 extends from the distal end 438 of the first piston 430 and is connected to a proximal end 440 of the second piston 434. When the first piston 430 advances, the piston rod 436 exerts a force on the second piston 434, causing the second piston 434 to also advance.

The first barrel 412 includes a port 442 toward the distal end 416 of the first barrel 412. A second tubing 450 may be connected to the port 442 so that a pressure may be exerted through the port 442, as described below, to stop the flowable compound from being delivered through the outlet port 424.

The second piston 434 may be used to exert pressure on bone cement 448 or other flowable materials contained within the second cavity 428 of the delivery syringe 410 so that the bone cement may be delivered into a vertebra or other bone structure. This pressure may be created by delivering a pressurized compound, for example CO₂ gas or liquid CO₂ through the fluid communication port 418 (as discussed below) into the first cavity 420. As a result of the pressure, the first piston 430 may be advanced distally to cause the piston rod 436 and the second piston 434 to similarly advance distally. Generally, the cross section of the first piston 430 must be greater than the cross section of the second piston 434 so that the pressure will increase as the first piston 430 and the second piston 434 move distally, as previously discussed.

Pressure is delivered to the delivery syringe 410, by means of a pressure deliver system 471. The pressure delivery system 471 generally comprises a canister configured to hold a pressurized compound 474, a trigger 472, and a pressure valve 470.

The pressurized compound is delivered to the delivery syringe 410 through the first tubing 440. The first tubing 440 connects the pressure valve 470 to the fluid communication port 418 on the delivery syringe 410. The first tubing 440 may be connected to the pressure valve 470 with a connector 476 or may be permanently attached. Similarly, the first tubing 440 may be connected to the fluid communication port 418 by means of a connector (not shown) or it may be permanently affixed.

The second tubing 450 connects the pressure delivery system 471 to the port 442 on the distal end 416 of the first barrel 412 of the delivery syringe 410.

The canister 474 is attached to the pressure valve 470 that controls the release of the pressurized compound into the delivery system (as described previously). When the trigger 472 is depressed, the pressure valve 470 is opened, the pressurized compound flows through the pressure valve 470, is pressurized, and released into either the first tubing 440 or the second tubing 450. Assuming the pressurized compound flows through the first tubing 440, a pressure is exerted on the first piston 438 causing the first piston 438 to move distally in the first barrel 412 which causes the second piston 434 to also move distally, as previously described, forcing the bone cement 448 out the outlet port 424. If the pressurized compound flows through the second tubing 450, which is connected to the port 442, a pressure is exerted such that the first piston 430 moves proximally in the first barrel 412 causing the second piston 434 to similarly more proximally in the second barrel 422. As a result of the first and the second pistons 430, 434 moving proximally, the flowable compound ceases flowing from the outlet port 424 and delivery to the cannula 102 and into the vertebra is stopped. The direction of the pressurized compound that is, the tubing through which it flows, may be determined by an additional valve (not shown) on the pressure valve 470, alternatively, the direction of the pressurized compound may be controlled by the position of the trigger 472.

As noted previously, the forgoing descriptions of the specific embodiments are presented for purposes of illustration and description. They are not intended to be exhaustive or to limit the invention to the precise forms disclosed and obviously, many modifications and variations are possible in view of the above teachings. The embodiments were chosen and described in order to explain the principles of the invention and its practical applications, to thereby enable those skilled in the art to best utilize the invention and various embodiments thereof as suited to the particular use contemplated. It is intended that the scope of the invention be defined by the following claims and their equivalents.

What is claimed:

1. An apparatus for delivering a flowable compound into a vertebra, comprising:
a cannula comprising a proximal end, a distal end having a size and shape for insertion into a vertebra, and a lumen extending between the proximal end and an opening in the distal end;
a delivery device comprising a barrel defining a cavity for receiving a flowable compound therein, a distal end comprising an outlet communicating with the cavity, the distal end being pivotally connected to the proximal end of the cannula such that the outlet communicates with the lumen of the cannula; and
a pressure delivery device in communication with the delivery device, wherein the pressure delivery device provides an actuating force that acts upon the flowable compound.

2. The apparatus of claim 1, wherein the actuating force indirectly acts upon the flowable compound.

3. The apparatus of claim 1, wherein the actuating force acts upon a piston disposed between the pressure delivery device and the flowable compound.

4. The apparatus of claim 1, wherein the actuating force acts directly upon the flowable compound.

5. The apparatus of claim 1, wherein the actuating force acts upon a piston configured to translate the actuating force through a medium to the flowable compound.

6. The apparatus of claim 5, wherein the medium is saline.

7. The apparatus of claim 1, wherein the actuating force acts upon a first piston configured to translate the actuating force through a medium to the flowable compound.

8. The apparatus of claim 1, further comprising a trigger.

9. The apparatus of claim 1, further comprising a trigger connected to the pressure delivery device, wherein the trigger is configured to control the actuating force.

10. The apparatus of claim 9, wherein a position of the trigger determines an associated position of a valve configured to control the actuating force.

11. The apparatus of claim 9, wherein a first position of the trigger opens a valve configured to control the actuating force, and a second position of the trigger closes the valve.

12. The apparatus of claim 9, wherein a first position of the trigger opens a valve and directs the actuating force in a first direction, and a second position of the trigger opens the valve and directs the actuating force in a second direction.

13. The apparatus of claim 9, wherein the trigger releases the actuating force and a valve connected to the pressure delivery device controls the actuating force.

14. The apparatus of claim 1, wherein the actuating force is CO₂

15. The apparatus of claim 1, wherein the actuating force is liquid CO₂.

16. The apparatus of claim 1, wherein the pressure delivery device further comprises a valve.

17. The apparatus of claim 16, wherein the valve is a blow-off valve.

18. The apparatus of claim 1, further comprising a valve proximal to the delivery device.

19. The apparatus of claim 18, wherein the valve is a manually controlled pressure relief valve.

20. The apparatus of claim 1, wherein the delivery device further comprises a valve, wherein the valve is designed to relieve pressure manually.

21. The apparatus of claim 16, wherein the valve controls the flow of the flowable compound.

22. The apparatus of claim 1, wherein the flowable compound is a bone cement.

23. An apparatus for delivering bone cement into a vertebra, comprising:
a cannula comprising a proximal end, a distal end having a size and shape for insertion into a vertebra, and a lumen extending between the proximal end and an opening in the distal end;
a delivery device comprising a barrel defining a cavity for receiving a flowable compound therein, a distal end comprising an outlet communicating with the cavity, the distal end being pivotally connected to the proximal end of the cannula such that outlet communicates with the lumen of the cannula;
a pressure delivery device in communication with the delivery device, wherein the pressure delivery device provides a gaseous actuating force that acts upon the flowable compound; and
a trigger connected to the pressure delivery device, wherein the trigger is configured to control the gaseous actuating force.

24. The apparatus of claim 23, wherein the pressure delivery device is a manual force generator.

25. The apparatus of claim 23, wherein the gaseous actuating force is CO₂.

26. The apparatus of claim 23, wherein the gaseous actuating force is liquid CO₂.

27. A pressure delivery device comprising:
a canister configured to hold a pressurized compound;
a valve connected to the canister and configured to control the pressurized compound; and
a trigger integrated with the valve, wherein a position of the trigger directs the pressurized compound.

28. The pressure delivery device of claim 27, wherein the pressurized compound is CO₂.

29. The pressure delivery device of claim 27, wherein the pressurized compound is liquid CO₂.

30. The pressure delivery device of claim 27, wherein the position of the trigger selects a tubing through which the pressurized compound flows.

31. A method for delivering bone cement into a vertebra, comprising:
inserting a cannula into a vertebra;
providing an actuating force from a pressure delivery device to a flowable compound, wherein the actuating force controls a movement of the flowable compound; and
delivering the flowable compound to the cannula and into the vertebra.
